(19) United States
(12) Patent Application Publication
(10) Pub. No.: US 2010/0055673 A1
(43) Pub. Date: Mar. 4, 2010

(54) TRANSPARENT MICROFLUIDIC DEVICE

(75) Inventors: Ajay Agarwal, Singapore (SG); Narayanan Balasubramanian, Singapore (SG); Lihui Guo, Singapore (SG); Chee Chung Wong, Singapore (SG)

Correspondence Address: CONNOLLY BOVE LODGE & HUTZ LLP, 1875 EYE STREET, N.W., SUITE 1100, WASHINGTON, DC, 20006, US

(73) Assignee: AGENCY FOR SCIENCE, TECHNOLOGY AND RESEARCH, Singapore (SG)

(21) Appl. No.: 12/303,112

(22) PCT Filed: May 31, 2006
(86) PCT No.: PCT/SG2006/000137

§ 371 Date: Nov. 19, 2009

(51) Int. Cl. C12Q 1/70; C12M 1/34; B05D 3/00; G01N 33/53; G01N 27/00; B01L 3/00

(52) U.S. Cl. 435/5; 435/287.1; 427/299; 435/7.2; 436/86; 436/94; 422/82.01; 422/102

(57) ABSTRACT

A device for analysing the status of a biological entity. The device (10) comprises a substantially transparent base substrate (11) having a recess defined therein by at least two opposing lateral walls and a base wall, a substantially transparent filler member (14) having at least a portion thereof occupying the recess, a substantially transparent separation layer (12) disposed between the filler member and the base substrate, and a channel (16) defined in the filler member, wherein the channel comprises an inlet and an outlet, the inlet being arranged on a first lateral wall of the filler member, and the outlet being arranged on a second lateral wall of the filler member, said first lateral wall of the filler member being arranged in opposing relationship with the second lateral wall of the filler member, and at least a portion of the first and the second lateral walls of the filler member being at least substantially perpendicular to the opposing lateral walls defining the recess.

The present invention relates generally to the field of microfluidics, and more particularly to microfluidic and nanofluidic systems that are substantially optically transparent, thus being adapted for applications that involve visual inspection of processes occurring within the system. Methods of fabricating such a system and for analyzing biological samples in a self-contained biochip platform are also disclosed.

### BACKGROUND OF THE INVENTION

Miniaturized devices for conducting chemical and biochemical analysis, otherwise known as microfluidic chips or biochips, have gained widespread acceptance as a standard tool for carrying out analytical and research purposes. The efficiency of these devices in automating repetitive laboratory tasks and their ability to provide highly sensitive levels of detection at a fraction of the cost as compared to traditional methods involving a highly qualified personnel and bulky equipment, has resulted in their widespread use in many types of applications. For example, microfluidic lab on chips are utilized as tools for conducting capillary electrophoresis and molecular diagnostics in a reproducible and effective manner. Microarrays or biochips are used to conduct hybridization assays for sequencing and other nucleic acid analysis.

Regardless of the application, the core of these devices is typically a structure that is formed from silicon. Silicon has been a material of choice for fabricating microfluidic devices because silicon process technology is capable of defining micro- and nano-structures precisely and predictably. By applying photo-masking and etching processes known from silicon chip manufacturing, it is possible to fabricate microfluidic devices that combine optical, fluidic, mechanical and electrical elements on a silicon wafer.

Most of the applications, which are carried out with these microfluidic devices typically require visual inspection of processes occurring within the device. For example, a patch clamp microfluidic device needs to be placed under a microscope in order to observe the proper patching of a micropipette tip against the surface of a cell. To facilitate visual inspection, the device should preferably be transparent so that it can be placed against a bright background (e.g. by illuminating the microfluidic chip from beneath using a fluorescent lamp). Due to silicon being an opaque material, silicon-based microfluidic chips cannot be viewed in such a manner, thereby limiting observation to situations when the observation is being carried out in a brightly lit environment.

The need for transparent materials in microfluidic devices also becomes apparent in patch clamp devices used for trapping living cells to record electrical activity of the cell membrane for purposes of drug discovery or other inter cellular interactions. Various types of microfluidic patch clamp devices that have been developed for this purpose typically have integrated sensing circuitry, microfluidic channels and other structures for automating the process of patch clamping.

Current fabrication technology relies chiefly on silicon wafer processing technology to create lateral channels. The problem is encountered, as mentioned above, that the starting material, typically a silicon wafer, is an opaque material through which light is unable to pass and thus presents limitations of use when optical observation is to be carried out with the device.

Various other schemes have also been used for the fabrication of micro-sized fluidic channels, which are integrated with other components like reservoirs, mixers, filters in transparent substrates. Since the patterning of glass wafers using standard projection photolithography is not possible, contact or proximity lithography has been used. However, this technique generally allows only the formation of relatively large channels.

Femtosecond ultrafast lasers have also been used to carry out ablation of glass. For example, it is possible to cleave minute glass fragments at various angles, drill small holes, and shape forms on the end or on the side of other glass pieces. However, with this approach it is not possible for forming lateral channels monolithically within a block of material.

McCreedy (Analytica Chimica Acta 427 (2001) 39-43) discloses a method for producing channels with a minimum width of 50 μm in glass wafers. Glass that is precoated with a thin chromium layer and photoresist is exposed to UV radiation using a standard UV exposure unit. The glass is then immersed in developer solution. Once the glass has been patterned, they are hard baked and then etched to form the channel.

Manor et al. (IEEE Sensors Journal, 3(6), 2003, pg. 687) discloses a method of channel fabrication in Pyrex and soda lime glass wafers. Fluidic channels were patterned on Pyrex after UV exposure on spin-coated SU-8 photoresist and adhesion promoter. After hard baking, wet etching was carried out using a solution of H₃PO₄, HF, and HNO₃. In the soda lime wafers, channels were etched to achieve 60 μm deep channels.

Joo et al. (Proc. MEMS '95, 1995, 362-367) describes the formation of metallic microchannels using the non-conformal deposition profile during nickel electroplating on a photoresist mold. Self-sealing metallic microchannels with a cross sectional area of 10×40 μm² were formed. As metal is a good thermal conductor, the metallic microchannel structure is used for the cooling of microelectronic devices.

U.S. Pat. No. 5,234,594 discloses a microchannel filter array formed by inserting an acid etchable glass rod into an inert hexagonal glass tube with inner dimensions that can accommodate the glass rod. By fusing the two glasses structures at close to melting point and then drawing them into fine filaments, it is possible to achieve glass channels with small diameters in the nanometre scale.

Seo et al. (Applied Phy. Lett. Vol. 84 No. 11, page 1973-1975) describes an integrated multiple patch clamp array chip which utilises lateral cell trapping junctions comprising patch channels arranged within opaque silicon wall that separates a cell reservoir from a suction chamber. Sample fluid is drawn from the cell reservoir into the suction chamber, and suction force immobilises cells onto the entrance of the patch channels. The chip was fabricated from PDMS micro-moulding and produced only semi-circular apertures. One shortcoming of a non-circular patch aperture is that high seal resistances that are in the range of giga-ohms cannot be attained, resulting in measurements in which background noise signals contribute to a significant portion of the measurements. Furthermore, the fabrication method is not adaptable towards transparent materials such as glass.

An object of the present invention is to provide a microfluidic device which deals with some of the drawbacks of the prior art devices, for example by providing microfluidic channels that are formed in transparent materials and methods which enables channels with nearly circular cross section and dimensions in sub-microns to microns range.

### SUMMARY OF THE INVENTION

According to a first aspect of the present invention, a microfluidic device is provided. The microfluidic device comprises a substantially transparent base substrate having a recess defined therein by at least two opposing lateral walls and a base wall. A substantially transparent filler member has at least a portion thereof, which occupies the recess. A substantially transparent separation layer is disposed between the base substrate and the filler member and a channel defined within filler member. The channel comprises an inlet and an outlet, the inlet being arranged on a first lateral wall of the filler member, and the outlet being arranged on a second lateral wall of the filler member. The first lateral wall of the filler member is arranged in opposing relationship with the second lateral wall of the filler member. At least a portion of the first and the second lateral walls of the filler member is at least substantially perpendicular to the opposing lateral walls defining the recess.

In another aspect, the invention also provides for a microfluidic device comprising a first fluid chamber for containing a particle to be tested, and a second fluid chamber that is fluidly connected to the first fluid chamber by means of a channel element in accordance with the first aspect of the invention.

The third aspect of the invention is directed to a method of fabricating the device of the invention. This method comprises providing a substantially transparent base substrate, forming a recess in a surface of the base substrate, forming a substantially transparent separation layer on said surface of the base substrate, filling said recess with a substantially transparent filling material, and subjecting the filling material to a condition that causes it to deform such that a channel is formed in the filling material.

According to a fourth aspect of the invention, there is provided a method of analyzing the status of a biological entity. The method comprises introducing the biological entity into the first fluid chamber of a microfluidic device in accordance with the invention. A first (reference) electrical signal that is associated with a first status of the biological entity is first obtained. The biological entity is then exposed to a condition (stimulus) that is suspected to be capable of changing its status. A second electrical signal that is associated with the status of the biological entity after exposure to the condition is taken. This second electrical signal can be analysed against the first electrical signal, where necessary, to determine the status or condition of the biological entity so as to determine the effect of the condition on the biological entity, for example.

The present invention provides means to fabricate transparent microfluidic channel structures with small dimensions that are beyond the reach of conventional micromachining techniques. The inventors have found that these microfluidic channel structures can be formed by using a combination of deposition and thermal reflow, in which a self-sealing phenomenon occurring during non-conformal deposition is used to generate a pre-cursor void structure, and thereafter, thermal reflow is used to shape the void structure into a channel according to a desired geometry.

Advantageously, forming the channel structure in a transparent material facilitates optical analysis, e.g. any device incorporating the channel structure can be placed over a light source for viewing of processes that take place within the device. An additional advantage of the present invention is that rounded channel inlets can be formed. In the context of patch clamp applications, for example, circular geometries are known to be capable of providing seal resistances that are in the order of giga-ohms, thereby reducing background noise signals and thus enabling more accurate patch clamp measurements to be taken. Furthermore, the present invention provides channels with dimensions ranging from a few micrometers to sub-micrometer levels, and are thus suited be used for a wide variety of applications involving biological samples ranging from cells, bacteria, virus, protein, and DNA molecules. From a fabrication perspective, by eliminating the need to carry out an etching step that is conventionally carried out in the fabrication of microfluidic channels, fewer steps are required for making the channel. Other advantages include the ease of packaging the device by means of a capping layer which contains microfluidic input and output channels and ports, and scalability to achieve a high-density array suitable for large scale parallel testing, since the micro-partitions in which the lateral patch channels are formed do not take much space and the profile of the channels to be formed in the partitions can be defined lithographically, unlike diaphragms used in existing planar patch clamps. Permits easy integration with other microfluidic unit operations modules such as micromixers and micropumps, for example.

The present invention is applicable to any type of fluids, including pure liquids, solutions, mixtures, as well as fluids containing particles such as suspensions, colloidal systems, colloidal solutions, or colloidal dispersions. The term ‘particles’ include small particles having a size in the range of several millimetres to less than 1 micrometer. In this context, the term ‘particle’ includes both inorganic particles (such as silica micro-spheres and glass beads) and organic particles. Organic particles would include biological entities, which in this context refers to biological material such as peptides, proteins, DNA, viruses, tissue fragments, single cell organisms such as protozoans, bacteria cells and viruses, as well as multi-cell organisms, single cells and subcellular structures. Cells to which the invention can be applied generally encompasses any type of cell that is voltage sensitive, or cells that are able to undergo a change in its electrical potential, including both eukaryotic cells and prokaryotic cells. Examples of eukaryotic cells include both plant and animal cells. Examples of some animal cells include cells in the nervous system such as astrocytes, oligodendrocytes, Schwann cells; autonomic neuron cells such as cholinergic neural cell, adrenergic neural cell, and peptidergic neural cell; sensory transducer cells such as olfactory cells, auditory cells, photoreceptors; hormone secreting cells such as somatotropes, lactotropes, thyrotropes, gonadotropes and corticotropes from the anterior pituitary glands, thyroid gland cells and adrenal gland cells; endocrine secretory epithelial cells such as mammary gland cells, lacrimal gland cells, ceruminous gland cells, eccrine sweat glands cells, and sebaceous gland cells; and other cells including osteoblasts, fibroblasts, blastomeres, hepatocytes, neuronal cells, oocytes, Chinese hamster ovary cell, blood cells such as erythrocytes, lymphocytes or monocytes, muscle cells such as myocytes, embryonic stem cells. Mammalian cells are an important example, being used in the screening of drugs. Other examples of eukaryotic cells include yeast cells and protozoa. Examples of plant cells include meristematic cells, parenchyma cells, collenchyma cells and sclerenchyma cells. Prokaryotic cells applicable in the invention include, for example, archaea cells and bacteria cells. The term biological entity additionally encompasses other types of biological material such as subcellular (intracellular) structures such as the nucleus, nucleolus, endoplasmic reticulum, centrosome, cytoskeleton, Golgi apparatus, mitochondrion, lysosome, peroxisome, vacuole, cell membrane, cytosol, cell wall, chloroplast, and fragments, derivatives, and mixtures thereof.

The microfluidic device according to the invention comprises a base substrate having a recess defined therein. The recess is present in the surface of the base substrate, defined by at least two opposing lateral walls and a base wall. Depending on the configuration desired, the recess may be defined (laterally) across the entire length/width of the base substrate (i.e. from one edge to another edge), or it may be defined near one edge of the base substrate. Also possible is that it is defined near the middle portion of the substrate so as to accommodate the fabrication of other fluid structures around it on the base substrate. In certain embodiments, for example where a through-recess spanning the entire surface or length (e.g. from end to end) of the base substrate is required, the recess is bounded by 1 pair of opposing lateral walls formed along the length of the channel and a base wall, while the ends of the recess are lateral openings not bounded by any lateral wall. Where the recess is formed to have one end defined at one edge of the base substrate and the other end terminating away from the edge of the base substrate (e.g. at the middle portion), then the recess is bounded by 1 pair of opposing lateral walls, 1 base wall, and 1 lateral wall connecting the two opposing lateral walls and opposing a lateral opening. Where the recess is defined entirely within the base substrate, the recess is then defined by 2 pairs of opposing lateral walls and a base wall.

The recess may have any suitable shape, such as a cuboid shape (e.g. rectangular or square shaped), in which case the recess is in the shape of a trench. Alternatively, it may be semi-cylindrical or hemispherical, or any other suitable irregular shape. Regardless of the shape, the depth of the recess may range from about 0.1 μm to about 10 μm if small channels are desired, or more typically between about 6 to 8 μm. For some embodiments requiring large channels or for achieving certain reflow characteristics with certain types of filling materials, a recess depth of more than 10 μm may be used to accommodate the larger channel. Apart from the depth, the width of the recess may also be sized according to the diameter of the channel it is required to accommodate, and may range from about 0.2 microns to about 5 microns.

Where a hemispherical, or semi-cylindrical shaped recess is formed in the base substrate, it is to be noted that the recess is then defined by a continuous wall. In this case, any two directly opposing end portions of the hemispherical walls of the recess may be considered to be any opposing lateral walls in the recess as defined by the plane of the openings at each of the two ends of a straight channel. The same applies to an irregularly shaped recess.

The recess present in the substrate serves to receive filling material for forming a filler member within the recess. The filler member is arranged such that at least a portion of it occupies the recess. This means that the filler member may be entirely present within the recess, or a portion of it may extend outside of the recess to cover a part of the top surface of the base substrate. Typically, deposition of filling material into the recess to form the filler member results in some of the filling material being deposited outside of the recess. If preferred, this extraneous filling material may be removed so that the filling material is present only within the recess.

The filler member has defined therein one or more microfluidic channels through which a fluid may flow and which are formed along the recess. The channel(s) terminates in an inlet at one end and an outlet at the other end, each of which are arranged on the opposing lateral walls of the filler member. These lateral walls of the filler member may be arranged flush with the exterior lateral surfaces of the base substrate, and together they define the lateral sides of the device. As will be described below, each lateral side may face a fluid chamber, being one of the walls that surround a fluid chamber. The opposing lateral walls of the filler member are orientated to be at least substantially perpendicular (also understood by the term ‘orthogonal’) to the opposing lateral walls defining the recess. In this manner, the orientation of at least the inlet aperture, or the outlet aperture as well, formed on these lateral walls of the filler member is such that the plane of each aperture is at least substantially vertical, thereby being substantially upright lateral apertures that are formed on the lateral walls of the filler member. By the term ‘substantially perpendicular’, it is meant that the angle between the plane of the opposing lateral walls of the filler member may be arranged not exactly at 90° to the plane of the opposing lateral walls defining the recess. The angle may deviate from 90°, as long as a part of the opening of the aperture is accessible horizontally.

For channels with small diameters, they may be situated entirely within and along the recess, meaning that the entire cross-section of the channel is located in the portion of the filler member found within the recess. However, for channels with diameters larger than the width or the height of the recess, they may not be fully accommodated within the recess, so a portion of the channel may be located partially outside of the recess. In yet other embodiments, for example in the case of channels with very large diameters relative to the size of the recess, the channels may be arranged entirely outside of and above the recess.

In one embodiment, the cross-section of at least a portion of the channel is at least substantially circular in shape. The term ‘at least substantially circular’ as applied to the cross-sectional shape of the channel includes any geometrical form that covers a 360° angle at the opening and thus means that it may be perfectly circular, or it may be, for example, elliptical or oval in shape (See FIGS. 1B, 1C and 1D). As it is desirable to achieve substantially circular apertures, fluid chambers, as described below, can be formed to coincide with this circular cross-sectional portion of the channel so that a circular aperture opening up into the fluid chamber is achieved. At least the first (inlet) aperture is preferably at least substantially circular in shape; in other embodiments, both the first (inlet) aperture and the second (outlet) aperture may be at least substantially circular in shape.

Depending on the application for which the device is intended, the dimensions of the first and the outlets may be varied to control the flow of substances within the channel. The selection of typical sizes for various types of substances is within the knowledge of the skilled person. For example, for carrying out electrophoresis of peptides, proteins, DNA, viruses, and bacteria in various separation media, the diameter of a channel may be made to conform to the size of these substances, e.g. in the sub-micron region. For patch clamp applications, the aperture may be adapted to be sufficiently small so as to achieve an effective seal on the surface of a sample particle or biological entity through the application of a suction force. If the sample biological entity is a human egg cell having a diameter of about 100 μm, the aperture that is used for performing the patch clamp can have a diameter of between about 0.1 μm to about 20 μm, or more preferably, about 1 μm to about 3 μm. Correspondingly, for even larger samples, the diameter may be beyond 20 μm. For smaller cells such as red blood cells, which typically have a diameter of about 5 μm, the aperture can have a smaller diameter of between about 0.1 to about 1 μm, if necessary. The diameter of the first and the outlets may be the same or different. In patch clamp applications, it is not necessary for both apertures to be circular in shape but it is preferred that the inlet is substantially circular to achieve an effective patch seal over the cell sample. The outlet aperture may therefore assume any other shape, since it is not used for patch clamping. Where only one aperture is at least substantially circular in shape, this aperture is preferably arranged to face the fluid chamber that is to be used for containing the sample particle, namely the first fluid chamber. In embodiments in which both the first and the corresponding outlets are at least substantially circular in shape, either aperture can then serve as the inlet for patch clamping the sample biological entity.

The channel connecting the first (inlet) aperture to the second (outlet) aperture may not have the same shape or size as the inlet/outlet. This may be due to various factors, such as irregularities in the fabrication procedure, non-uniform conditions across the length of the channel during fabrication and uneven deposition of filling material into the recess when forming the filler member. Any suitable cross-sectional shape, for example circular, elliptical or rectangular shapes, would be possible. The diameter of the channel and the diameter of the inlet and outlet to the channel are both generally similar, though slight differences in size are also acceptable. Commonly, the channel has the same cross-sectional shape as one or both apertures. The channel is preferably arranged laterally within the filler member, i.e. within the horizontal plane of the base substrate. Sections of the channel may slope upwards or downwards within the filler member due to uneven deposition of filling material into the recess when forming the filler member. The longitudinal or axial length of the channel may be orientated to be in alignment with the length or width of the recess. In one embodiment, the channel has a length of between about 1 μm to more than 100 μm; the channel may also have a channel diameter of between about 5 μm to about 20 μm.

In order to achieve a fully transparent device, components comprised in the device are preferably substantially transparent, i.e. allows visible light to pass through. In one embodiment, the base substrate of the partitioning element comprises silicon dioxide in crystalline and/or amorphous forms. Crystalline forms of silicon dioxide have SiO₂ molecules arranged in a specific order throughout, whereas amorphous forms do not have long-range order. All deposited and thermally grown silicon dioxide in semiconductor processing are known to be amorphous. Examples of suitable crystalline forms including quartz, tridymite and cristobalite; examples of amorphous forms include fused silica and fumed silica. Other transparent materials that have been contemplated for use as the base substrate includes materials comprising transparent alumina, with some examples being ruby and sapphire.

Without wishing to be bound by theory, the inventors have found that non-conformal deposition of filling material into the recess followed by its thermal reflow requires that the filling material preserve its ability to deform/reflow throughout the period of heating. By having a separation layer interposed between the filler member and the base substrate, it is found that interaction between the filler member and the base substrate is minimised. This interaction may include, for example, the diffusion of dopants, which could be present in the filler member for reducing its glass transition temperature into the base substrate. At the level of several microns, this interaction has been found to have a significant impact on the reflow characteristics of the filler member. In addition, with the separation layer present, self-sealing circular micro channels with large cross-sections could be formed from the thermal reflow process. It is believed that this phenomenon is caused by released of stress built up within the 2 layers during the thermal annealing process.

Fabrication of a substantially transparent separation layer is achieved by depositing the separation layer on the top surface of the base substrate, including the exposed surfaces of the recess, prior to filling the recess with filling material. The separation layer serves as a diffusion barrier between the base substrate and the filler member to prevent or retard the diffusion of dopants from the filler member to the base substrate. To provide good diffusion barrier properties, the diffusion barrier is preferably a material that exhibits minimal reactivity, or ideally, complete inertness, towards materials adjacent to it. The separation layer may thus comprise any compatible inert material that is impermeable to ionic or atomic diffusion especially at high temperatures of several hundred degree Celsius, such as silicon-based ceramic materials like silicon nitride or silicon carbide. Poly-silicon and amorphous silicon are other possible materials as they are translucent when deposited as a thin layer. Where the filler member comprises materials which are doped with lattice-disrupting dopant atoms, such as silicon dioxide doped with boron or phosphorous, the separation/barrier layer serves to prevent the diffusion of dopant atoms from the filler member into the base member. Especially at high temperatures, dopants from the filling material have a tendency to diffuse into base substrate if both filler member and base substrate are in direct contact. If the dopants are allowed to diffuse away from the filling material and the filling material is allowed to become depleted of dopant atoms, the filling material would lose its reflow property, with the result that the filling material cannot reflow properly to transform the initial triangular void into a circular channel or any other desired channel geometry.

In order to bring about a reflow of the filler member within the recess of the base substrate, the base substrate preferably has a higher glass transition temperature than the filler member so that upon heating above the glass transition temperature of the filler member and above that of the base substrate, the base substrate still retains its form, while the filler member deforms and reflows within the recess to form a desired geometry. In some embodiments, the filler member comprises a dielectric material and more preferably, the dielectric material comprises a doped glass, e.g. doped silicon dioxide. Examples of doped glasses include halogen-doped glass, transition metal-doped glass, spin-on-glass (SOG), phospho-silicate glass (PSG), boro-silicate glass, boro-phospho-silicate glass (BPSG), or soda lime glass.

The present invention is applicable for any microfluidic systems in general, and the aforementioned embodiments may be collectively called a channel element and can be used to fluidly connect two fluid chambers. The term “channel element” is used interchangeably with other equivalent terms. For example, if the device is to be used in a patch clamp device, the channel element may be termed differently. Taken in the specific context of a patch clamp, the aforementioned embodiments of the device of the invention may be directed to a partitioning element (hereinafter used interchangeably with the term ‘partitioning wall’) comprising the lateral channel with lateral apertures (i.e. the inlet and the outlet to the lateral channel) and which is used to separate two fluid chambers in a lateral patch clamp device.

In other embodiments, the device of the invention may comprise a first fluid chamber that is separated from a second fluid chamber by the partitioning element, the first fluid chamber being in fluid communication with the second fluid chamber via the channel present in the filler member. Fabrication may be carried out by first forming the partitioning element and then assembled into a separate fluid chamber member to obtain the lateral patch clamp device. Alternatively, the first fluid chamber and the second fluid chamber may be monolithically defined in the base substrate at, respectively, the inlet aperture and the outlet aperture of the channel. In this manner, a lateral patch clamp comprising two fluid chambers connected through the channel in the filler member is realized.

In some embodiments, both the first fluid chamber and the second fluid chamber are similar (identical) in shape, dimension and/or geometry. Alternatively, the first and the second fluid chambers may be different in shape, dimension and/or geometry. The first fluid chamber that is used for containing the sample biological entity may be a closed/isolated chamber or an open chamber fluidly connected to other fluid channels or a supply chamber. In a presently preferred embodiment, the first fluid chamber is fluidly connected to a fluidic channel that is fluidly connected to a source supplying the sample. The second fluid receives fluid from the first fluid chamber and may be fluidly connected to a drainage channel for discarding the sample.

Fluid chambers that are formed in the base substrate may be transferred onto a transparent capping substrate, e.g. glass, via a variety of means including anodic, fusion or adhesive bonding. The capping substrate may include a variety of fluidic structures such as fluidic chambers, and fluid channels, etc. Such a cover may be bonded onto the base substrate via a variety of means, such as anodic bonding. The cover serves to seal any open fluidic structure that has been defined in the base substrate. It may also have any number of input and output ports etched into the cover for introducing/removing fluids. If electrodes are not built into the device, they may be provided by an external measurement system, and may be arranged to be inserted into the fluid chamber via access ports if electrical measurements need to be done in the device.

In order to form a complete microfluidic system for carrying out a complete suite of analytical processes, the fluid chambers may be operably connected to at least one microfluidic unit operation module. The term “microfluidic unit operation module” refers to microfluidic structures, which can carry out unit operations of microfluidics, including mixing, separation, reaction, pumping, dispensing and sensing, for example. Modules capable of carrying out these unit operations include micro-dispensers, micromixers, micropumps, injectors, sensors, reservoirs, and reaction chambers.

For sensing applications, electrodes may be disposed in the first fluid chamber and the second fluid chamber for the purpose of taking electrical measurements between an upstream point and a downstream point of an immobilised particle or biological entity. Electrical measurements that can be taken include current flow (due to the flow of ions through the immobilised particle e.g. cell wall of an oocyte) as well as voltage potential, for instance. In the context of patch clamping applications, the electrode arranged in the upstream side of the immobilised particle may be termed a reference electrode, and the electrode arranged in the downstream side of the immobilised particle may be termed a sensing electrode. More than one reference electrode and one sensing electrode can be positioned within the channel, e.g. close to the immobilised particle, functioning either for sensing purposes or for stimulating/electrocuting or moving the immobilised particle or for altering conditions in the fluid chambers. If it is desired to observe the response of the sample biological entity to electrical stimulation, additional electrodes can be arranged on the partitioning element, for example, in order to the sample biological entity, thereby stimulating it electrically. Auxiliary circuitry (e.g. electro-physiological measurement circuitry), either integrated into the device or provided by an external measurement system, may be connected to these electrodes.

Other applications which the device can be used for includes electrophoresis or the filtering of particles. Filtering can serve a variety of purposes, including pre-concentrating a sample particle based on electrokinetic trapping (cf. Wang et al, Anal. Chem. 2005, 77, 4293-4299). Other examples of filtering applications include DNA sieving or the isolation of a virus sample, for instance. Filtering can be accomplished by placing a sample containing particles that are to be sieved out into the first fluid chamber. By applying a suction force in the channel present in the filler member, particles smaller than the diameter of the narrowest section of the channel will enter into the channel and be discharged into the second fluid chamber. Particles larger than this diameter are trapped and remain within the first fluid chamber

The device of the invention can be scaled up to process large quantities of the same or different samples simultaneously. For this purpose, the device may include a plurality of channels defined in the filler member, all of which are arranged in the portion of the filler member occupying a single recess. Alternatively, in a preferred embodiment, there may be a plurality of recesses defined in the base substrate and the filler member has corresponding portions thereof arranged in each recess. Each portion of the filler member occupying the recess may have defined therein a channel. A partitioning element comprising a plurality of channels may be used to separate a plurality of first and/or second fluid chambers, each of which is used to analyse a plurality of particles simultaneously.

In one embodiment, the device comprises one common first fluid chamber and a plurality second fluid chambers fluidly connected to the first fluid chamber via the plurality of channels in the partitioning element. In another embodiment, a plurality of inlets are formed in the first surface of the partitioning element, and a plurality of outlets are formed on the second surface of the substrate. Each inlet of the plurality of inlets is fluidly connected to a corresponding outlet of said plurality of outlets via a channel formed within the substrate, so that different samples can be placed within each individual first fluid chamber for simultaneous processing. In both embodiments, the second fluid chambers are isolated from each other to allow independent electrical recordings to be taken. To achieve this arrangement, the partitioning element may be bonded to the multi-well array such that each inlet of the plurality of inlets is in alignment with each individual first chamber of said plurality of first chambers. In a further embodiment, the device of the invention may comprise a plurality of partitioning elements, each of which is connected to a respective first fluid chamber constituting the multi-well array.

In one embodiment, the device additionally comprises a substantially transparent capping substrate. The capping substrate is included in the device as a capping structure, such as a glass cover, to seal up any open fluidic chamber in the base substrate. It may be in the form of a flat structure and/or a thick multi-layered structured having defined therein fluidic structures such as fluidic chambers, fluidic channels, fluidic input and/or output ports, etc. The capping substrate may be derived from any suitable substantially transparent materials such as glass or transparent polymers. The capping substrate may be attached to device by means of bonding, typically after the formation of fluidic chambers on two sides of the channel element and after opaque components of the handling substrate are removed. In the context of the specification, the term “capping substrate” may be used interchangeably with or is to be understood as being analogous to the terms “final handle substrate” and “final handle wafer”.

The invention also provides a method for forming a lateral patch clamp aperture having patch apertures that are at least substantially circular in shape, and with circular cross-section diameter in the range of microns to nanometres. The fabrication of lateral patch channels with circular geometries presents several problems. In particular, there are difficulties in achieving a patch inlets with a circular shape in a transparent material. A circular inlet is important for achieving a tight seal over the surface of a sample, e.g. a cell, through the application of a suction force.

In accordance with the invention, the method disclosed herein provides lateral channels which are formed in transparent materials and with substantially circular inlets. The method comprises first providing a substantially transparent base substrate and then forming a recess in a surface of the base substrate. The recess can be formed by any conventional means applicable for the base substrate material, such as wet etching. The dimensions of the recess can be varied according to the size of the particle to be analysed. In one embodiment, the width of the recess is between about 0.1 μm to about 20 μm, and the length is between about 1 μm to about 100 μm.

If base substrate comprises silicon dioxide, a variety of etching solutions can be used, depending on the type of silicon dioxide present. Silicon dioxide etching is intrinsically anisotropic due to the fact that the strong chemical bond between the silicon and oxygen requires ion bombardment to break. Different forms of silicon dioxide will therefore exhibit different reactivities towards different types of etching solutions. Typically, highly doped oxides etch faster and oxides with high carbon content etch dirtier. The chemical reaction for this etching process is given below:

SiO₂(s)+CF₄(g)+plasma--------->SiF₄(g)+CO(g)

After forming the recess, a substantially transparent separation layer is formed on the surface of the base substrate. This surface preferably includes the surface of the recess the defining the recess. For example, the separation layer may comprise 100 nm polysilicon or silicon nitride.

Filling material is subsequently deposited into the recess. Any filling material capable of deforming is suitable for this purpose. In one embodiment, the filling material comprises a doped silicon oxide material. The material preferably has a low glass transition material, or more preferably with a glass transition temperature that is lower than that of the base substrate material. The filling may be carried out for example by means of a deposition process. Examples of applicable deposition processes include plasma enhanced chemical vapour deposition, low pressure chemical vapour deposition, sub-atmospheric chemical vapour deposition, or physical vapour deposition. The filling material is deposited into the recess in such a way as to trap a void there within; in particular the void is to be trapped along the recess. In one embodiment, the recess is filled with filling material via a deposition process, which results in non-conformal surface topography of the filling material in the recess. In other words, the filling of the recess with a filling material comprises depositing the filling material into the recess in a manner that causes the filling material to pinch together at the opening end of the recess, thereby trapping a void in the filling material. The void extends laterally through the filler member from one end of the recess to the opposing end of the recess. The void contains the gas in which the deposition is carried out, typically being N₂ or O₂ for example.

In one embodiment, the filling material comprises doped silicon dioxide. Typically, filling is achieved by softening the filling material and letting it flow into the recess as a liquid, thereby minimizing surface tension and thus curvature. It is desirable to use such “flow” processes with dielectric layers to smooth out the rough edges of underlying features. However, as pure silicon dioxide requires high temperatures of about 1300° C. to about 1400° C. to flow readily, introducing dopants into the silicon dioxide can reduce the softening point (re-flow temperature) of the silicon dioxide. In some embodiments, phosphorus may be added to obtain phosphosilicate glass (PSG) that flows readily at 1000° C. for 6 to 8 weight % P in the alloy. Alternatively, boron may be added to obtain borosilicate glass (BSG) or boro-phosphosilicate glass (BPSG). Alternatively, borophosphosilicate glass (BPSG) can also achieve a lower flow temperature: typically around 900° C. for 4-5 wt. % of each dopant. Arsenic may also be employed as an additional dopant in each case. Apart from these materials, a further alternative is methylsiloxene, a Si—O polymer with attached methyl groups, commonly known as Spin-on-Glass (SOG). SOG is a material commonly used in fabricating silicon integrated circuits and is generally used as a planarizing layer to provide smooth surfaces for photolithography.

In order to prevent over-etching, the base substrate is preferably formed on an etch stop layer. In general, the etch stop layer may be selected on the basis of its differential etch rate in Si etching solutions. Any variety etch stop layer may be used for this purpose. For example, an etch stop layer comprising a Si-Gb alloy may be used, or one which comprises 100 nm thermal oxide and 150 nm of silicon nitride. More preferably, the etch stop layer is located on a conventional silicon wafer/chip obtainable from silicon foundries, including Czochralski (CZ) wafers, Float Zone (FZ) wafers, silicon epitaxial (SE) wafers and silicon on insulator (SOI) wafers.

After deposition is completed and a void is trapped in the filler member, the doped silicon oxide material is subsequently subjected to a condition that causes it to deform and reflow, thereby changing the shape of the void to eventually form a channel in the doped silicon oxide material. In general, the deformation procedure for forming the void in the recess depends on the width-to-depth ratio of the recess, profile of the recess, deposition pressure of the filler, etc. For example, it is possible to form the void by simultaneously depositing a uniform layer of the doped silicon oxide material over the lateral walls of the recess, and subsequently heating the doped silicon oxide material in order that it flows down into the base wall. The doped silicon oxide material around the opening of the recess pinches automatically due to stress relaxation, thereby enveloping a void beneath. The time required for heating the doped silicon oxide material to deform the doped silicon oxide material sufficiently to achieve an at least substantially circular aperture or channel depends on the initial void dimension, deposition conditions, heating temperature, heating pressure and final dimension of the aperture. It can vary from few minutes to few hours.

In one embodiment, the doped silicon oxide material is heated above its glass transition temperature, but below the melting point in order to bring about the deformation of the doped silicon oxide material without melting it. Temperature range at which heating is carried out may be between about 800° C. to about 1200° C. for time periods of between about 30 seconds to about 240 minutes. The pressure at which deposition takes place may be in the range of about 3 Torr to about 50 Torr, while reflow pressure is about atmospheric pressure.

Auxiliary structures may be formed around the channel, including fluid chambers, microfluidic channels, ports, and electrical circuitry may be integrated with the device. The formation of such structures is within the knowledge of the skilled person, and may be carried out, for example, via a combination of etching, deposition and bonding procedures for example.

The channel element (or partitioning element) can be fabricated independently, and then assembled with other components to form a complete device, or it may be formed monolithically without the need to assemble it with other components. In one embodiment, the channel element is fabricated independently on a handling substrate such as a silicon wafer or any other suitable material having an etch stop layer and having a sufficiently large size for easy handling. In the context of the specification, the term “handling substrate” may be used interchangeably with or is to be understood as being analogous to the terms “initial handle substrate” and “initial handle wafer”. After forming the channel element, it is transferred and bonded (through anodic, fusion or adhesive bonding, etc.) in a preliminary chamber that has been formed in a substantially transparent secondary base substrate (e.g. a glass substrate or a transparent polymer substrate), thereby dividing the preliminary chamber into two fluid chambers that are separated by the channel element. In another embodiment, monolithic fabrication is carried out in which the channel element is first fabricated in a base substrate of sufficiently large size, and subsequently, the first and second fluid chambers is formed monolithically in the base substrate, with the channel of the channel element connecting the two fluid chambers. In either fabrication methods, a substantially transparent capping substrate (e.g. glass or transparent polymer substrate as well) may be bonded over the channel element to cover the top of the fluid chambers. The capping substrate may have pre-drilled holes (for fluidic inputs and outputs) so that fluidic chambers may be accessed by electrodes for the measurements. Any opaque material present in the handling substrate should also be removed, either prior to bonding in a preliminary chamber or at the end of monolithic fabrication, in order to achieve a transparent completed device.

One advantage of the method of the present invention is that the channel cross section dimensions can be predicted and controlled through the selection of parameters for deforming the doped silicon oxide material used to form the filler member. Additionally, the process is CMOS compatible and hence can be integrated with other silicon technologies to realize other device components like electrodes, reservoirs, etc. Channel fabrication cost is low as no specialized tools/processes like electron beam lithography, wafer bonding and laser ablation. If desired, channels of different dimensions can be obtained within a single device by varying dimensions of the recesses formed on the surface of the base member. Hence, a single device can be used for analysing different sizes of cells/biological molecules. Furthermore, the channels can be easily formed in the partitioning element due to the ability of the channels to self-align during fabrication. Smooth oxide surface is retained so that side wall roughness is reduced and wafer bonding can be easily carried out.

The microfluidic device according to another aspect of the invention comprises a first fluid chamber for containing a sample to be tested, and a second fluid chamber that is separated from the first fluid chamber by a partitioning element according to the first aspect of the invention. The channel in the partitioning element is orientated such its inlet faces the first fluid chamber and its outlet faces the second fluid chamber, thereby fluidly connecting the first fluid chamber to the second fluid chamber.

The device according to this aspect represents the general form of a complete microfluidic chip which can be deployed at the end-user level to collect samples for analysis. This device may be obtained several ways as mentioned earlier, for example, by fabricating the partitioning element independently, and then assembling the partitioning element into a fluid chamber member, for example by bonding, or by forming a first and second fluid chambers monolithically into the base substrate with the recess with the filler member arranged between the two fluid chambers.

Various modifications can be implemented to make the chip more durable for physical handling and transportation. For example, the device may be provided with a capping substrate such as a glass lid/cover to cover the top of the filler member and the base substrate, as well as the top of the fluid chambers for sealing purposes. At the same time, any opaque component present in the handling substrate on which the channel element is formed initially, such as the silicon layer in a silicon wafer, may be removed. The chip may also incorporate one or more ports capable of receiving a delivery needle for introducing a sample into the first fluid chamber. For large scale testing, arrays of fluid chambers may also be connected via a plurality of channels to enable massively parallel testing to be carried out (e.g. screenings can be carried out simultaneously to determine the effect of many substances on a particle type of cell). In one commercial useful implementation, the device may be used in conjunction with a measuring system which takes readings from the device and which additional provides electrical sensing circuitry, suction force control, data collection, for example.

In one embodiment, an electrical measurement device is connected to the first fluid chamber and the second fluid chamber for determining one or more electrical characteristics of a test particle(s). The electrical measurement device may comprise a pair of electrodes connected to a current or voltage measurement equipment and which may each be inserted into the first fluid chamber and the second fluid chamber from access ports.

A further aspect of the invention is directed to the use of the device of the invention for analysing the status of a biological entity. In general, the biosensor of the invention may be used in any application requiring electrophysiological measurements of biological entities such as cells. Such applications typically require contact between the biological entity being evaluated and a current-sensitive sensor, such as a transistor or a conventional micropipette patch clamp or the sensing electrodes placed within the first and the second fluid chambers. Common applications for the biosensor include the screening of drugs (e.g. electrophysiological determination of compound activity on ion channels in cell membranes is studied) and studies into the characteristics of cells (studies on the mechanisms of microelectrode electroporation).

In the first step of the method, the biological entity is introduced into the first fluid chamber of a device in accordance with any suitable embodiment of the invention, namely, in accordance with the third aspect of the invention or in accordance with embodiments in accordance with the first aspect of the invention and which incorporate a fluid chambers.

A first (reference) electrical signal that is associated with a first status of the biological entity is recorded via sensing electrodes that are either integrated into the device or provided by an external measuring equipment. Thereafter, the biological entity is exposed to a condition or stimulus that is suspected to be capable of changing the status of the biological entity. Exposure to such a condition includes surrounding the biological entity with a chemical compound which is being evaluated for efficacy on the biological entity, in particular a chemical compound which has is suspected to be capable of modulating the ion channel behaviour on the biological entity; the term also includes electrically stimulating the biological entity.

After exposure to the condition, the biological entity changes, and a second electrical signal that is associated with the changed status of the biological entity after exposure to the condition is being measured. Measurements of the first and the second electrical signal prior to and after exposure to the condition may be carried out continuously, meaning that the electrical signals may be continuously monitored before the exposure to the condition, until after the biological entity exhibits the full extent of the effect of the condition on it.

In cell membrane studies, e.g. studies characterising membrane polarisation, or studies determining trans-membrane threshold potential for pore formation can be made by making a first measurement of the electrical signal of the environment upstream and downstream of the biological entity in order to determine the ion current flow through the biological entity. Subsequently, after having exposed the biological entity to a condition suspected of being capable of altering the status of the cell, a second measurement of the ion current is made and is compared to the first measurement. The differences between the first and the second measurement can be compared to values in existing literature to determine whether there is a status change in the biological entity before and after exposure to the condition. For example, the second electrical signal may be compared against a known electrical signal that is known to correspond to a changed status; alternatively, the magnitude of the difference between the first and the second electrical signal may be compared to the pre-determined threshold electrical signal values, so that when the magnitude of the difference between the first and the second electrical signal is larger than the magnitude of the pre-determined threshold electrical signal value, the condition to which the biological entity is exposed is determined to be deemed to be capable of changing its status.

Measurements of the first and/or second electrical signal may comprise measurements of electrical current passing through any type of transport structure located within or isolated from the region of the cell on which the suction force is applied. In accordance with conventional patch clamp techniques, the measurement may be carried out on an intact cell using the whole cell or cell attached approach, or on a fragment of a cell using the inside-out and outside-out approach. In this respect, transport structures in a cell include any of the following structures located in a cell membrane: anion channels, cation channels, anion transporters, cation transporters, receptor proteins and binding proteins. Measurement of the first electrical signal may comprise measuring a reference electrical potential of the sample solution containing the biological entity, said electrical potential being measured from a reference electrode present at the top surface of the biosensor and which is in contact with the sample solution.

In one embodiment, immobilization of the biological entity onto the biosensor is performed by means of suction force that is generated at the inlet as well as any other suitable types of forces such as dielectrophoresis. When a sample fluid is placed in the first fluid chamber, any suction force applied through the channel results in fluid being drawn through the channel, and then entering the inlet and subsequently draining through the aperture downstream of the channel, namely the outlet. By applying a sufficiently strong suction force, the particle is drawn towards the inlet and eventually becomes patched over the inlet, forming a seal over the edges of the aperture and thereby restricts the free flow of fluid and ions through the channel. This arrangement establishes a high electrical resistance seal over the aperture. This suction force can be generated by withdrawing fluid from the second fluid chamber by means of a syringe, for example. Suction force can also be generated via pump-driven suction of the sample solution containing the biological entity.

When using the device to carry out conventional patch clamp measurements on a biological entity, the sensing electrodes in the fluid chambers may be used both to control the current (current clamp) or voltage potential (voltage clamp) in each fluid chamber and to measure the ionic currents conducted across the biological entity or the membrane potential across the cell membrane of the biological entity. Measurements of the first electrical signal may comprise measuring an electrical current passing through at least one ion channel isolated within the region of the cell on which the suction force is applied.

If desired, optical analysis can be carried out to augment the electrical measurement analysis. For example, a visualization substance can be added to the first fluid chamber to assist a human operator to visually determine the status of the seal formed by the biological entity over the inlet. The visualization substance can be a colour dye, such as ethidium bromide or disodium fluorescein, for example. If the pigment is seen travelling into the second fluid chamber, then the seal is not formed effectively and another attempt must be made to immobilise the biological entity over the aperture.

Apart from patch clamp applications, the device of the invention can also be formed in a manner required for carrying out various other applications such as capillary electrophoresis or DNA sieving. The device can also be used to immobilize or filtering any type of small particle over the laterally arranged aperture located on the filler member. For example, the device can be used for filtering and for trapping certain types of biological entities such as viruses and pathogens. For filtering applications, the diameter of the inlet aperture can be in the sub-micron range. Application of suction force results in biological entities which are smaller than the aperture diameter to enter the aperture and then travel through the channel into the second fluid chamber, while large particles remain trapped within the first fluid chamber.

These aspects of the invention will be more fully understood in view of the following description, drawings and non-limiting examples.

### BRIEF DESCRIPTION OF THE DRAWINGS

In order to understand the present invention and to demonstrate how the present invention may be carried out in practice, preferred embodiments will now be described by way of non-limiting examples only, with reference to the accompanying drawings, in which:

FIG. 1A shows a cross-sectional view of a microfluidic device according to an exemplary embodiment; FIG. 1B shows a cross sectional view of a segment of the device showing a channel with diameter of about 1 μm formed within a recess in the base substrate; FIG. 1C shows a cross sectional view of a segment of the device showing a channel with a diameter larger than the width of the recess being formed partially above the recess; FIG. 1D shows a cross sectional image of a segment of the device showing a channel with diameter of more than 10 μm being formed above the recess.

FIG. 2 shows a transverse cross sectional view of another microfluidic device of the invention.

FIG. 3 shows a simplified diagram of a lateral patch clamp setup.

FIG. 4A shows a perspective view of a partitioning element having a plurality of channel; FIG. 4B shows a scanning electron microscope photograph of a cross section of the plurality of channels.

FIG. 5 shows a top view of a device of the invention in which a plurality of first fluid chambers and a plurality of second fluid chambers are each arranged in an array along the partitioning element. Each first fluid chamber is individually isolated from each other and connected to a respective second isolated fluid chamber via a channel.

FIG. 6 shows a top view of a device having an alternative layout in which only a single first fluid chamber is fluidly connected to a plurality of second fluid chambers.

FIG. 7 is a general flow diagram of the method of fabricating the device of the invention.

FIG. 8A to FIG. 8H depict a procedure for forming small channels within the recess of a base substrate, bonding it with glass substrate and removal of substrate silicon. FIG. 8I depicts a cross section of the product obtained from the procedure.

FIG. 9A to FIG. 9J depict a specific procedure for forming large channels arranged partially outside of the recess of a base substrate, bonding it with glass substrate and removal of substrate silicon. FIG. 9J depicts a cross section of the product obtained from the procedure.

FIG. 10 depicts the steps for forming a microfluidic device with metal electrodes shown in FIG. 2.

FIG. 11A to FIG. 11F show various 3D images of perspective views of an embodiment of the device as seen from scanning electron microscope photographs, showing in particular the circular aperture that is formed in the filler member. FIG. 11G shows a top view of the device depicted in FIG. 11D.

FIG. 12A and FIG. 12B show a close up portion of a channel under two different optical imaging modes.

### DETAILED DESCRIPTION

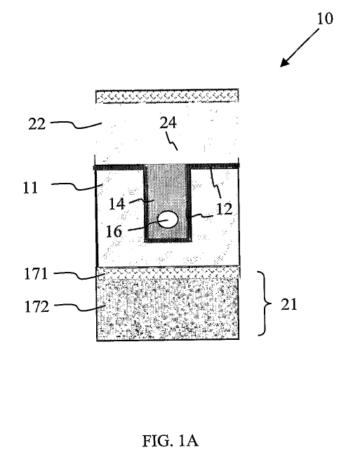

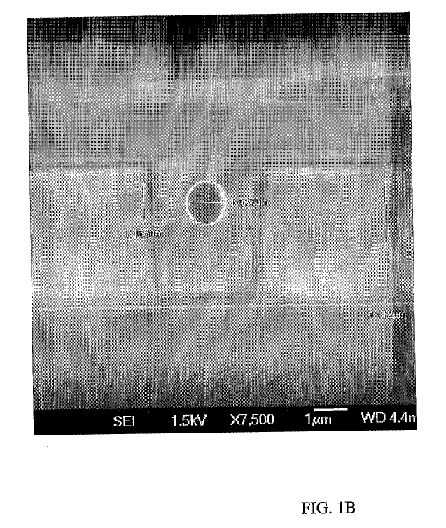

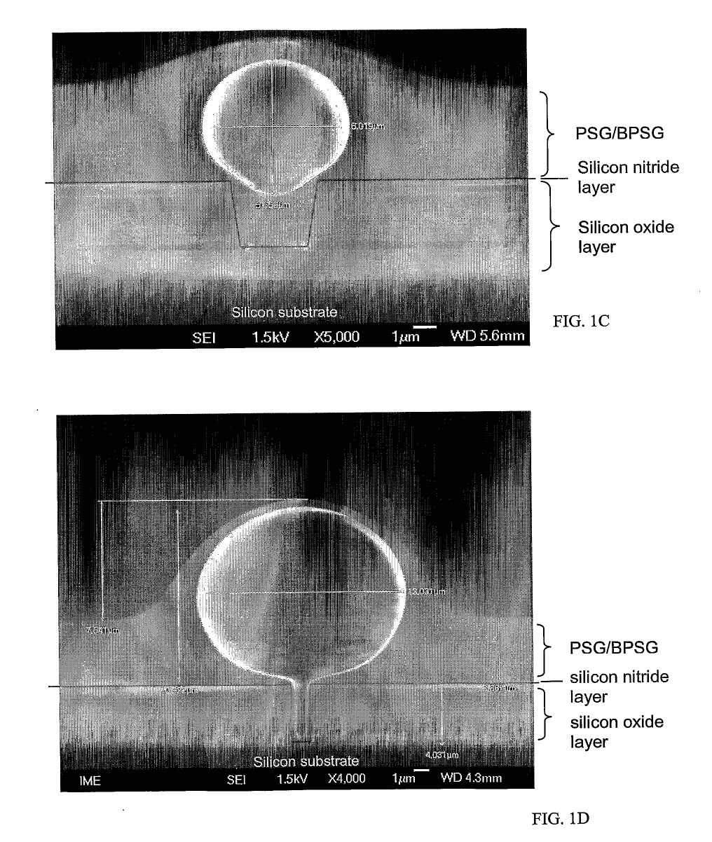

A cross-sectional side view through a microfluidic device 10 according to a first embodiment of the present invention is depicted in FIG. 1A. The device comprises a base substrate 11 having a recess (not labelled) that is occupied by filling material which forms a filler member 14. A separation layer 12 is disposed between the base substrate 11 and a filler member 14. The filler member 14 has a channel 16 defined therein, and in particular arranged in the portion of the filler member located in the recess. The base substrate is formed on an etch-stop layer 171 of a silicon wafer 21 that comprises the etch-stop layer 171 and a silicon layer 172. A transparent glass cap 22 having a chamber 24 is bonded over the filler member. FIG. 1B shows a scanning electron microscope (SEM) photograph of the cross-section of a channel element according to an embodiment of the invention. A channel with dimensions of about 1 μm is formed within a recess having a width of about 3 μm and height of about 4 μm. As can be seen from the figure, a separation layer comprising polysilicon is disposed between the filler member and the base substrate. FIG. 1C shows another cross-sectional image of a segment of the device showing an elliptical channel with diameter of about 5.6 μm to 6.0 μm formed substantially outside of a 3×3 μm recess. FIG. 1D shows another cross-sectional image of a segment of the device showing an elliptical channel having dimensions of 13.0 μm by 10.9 μm formed above a 1×3 μm recess. An elongated section of the channel is also formed within the recess.

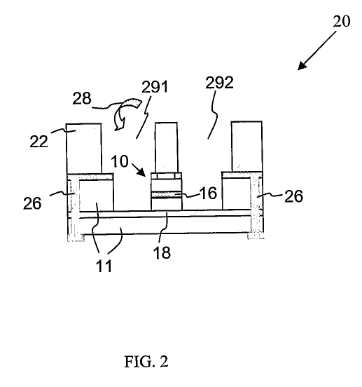

This microfluidic device 10 can be installed as a partitioning element when used to separate 2 fluidic chambers as shown in the following FIG. 2, which shows another cross-sectional side view of the same device taken at 900 from the view in FIG. 1, with the silicon layer 21 removed. It can be seen that partitioning element 10 is arranged between a first fluidic chamber 291 and a second fluid chamber 292. Channel 16 extends laterally within the partitioning element 10 to fluidly connect the first fluidic chamber 291 and a second fluid chamber 292. Electrodes 26 are provided in the vicinity of each fluid chamber 291, 292 to enable electrical measurements to be taken from each fluid chamber. If the top of the glass cap 22 is etched open to provide access into the fluid chambers 291, 292, sample solution can be added in the direction indicated by the arrow 28 into the first fluidic chamber 291, for example.

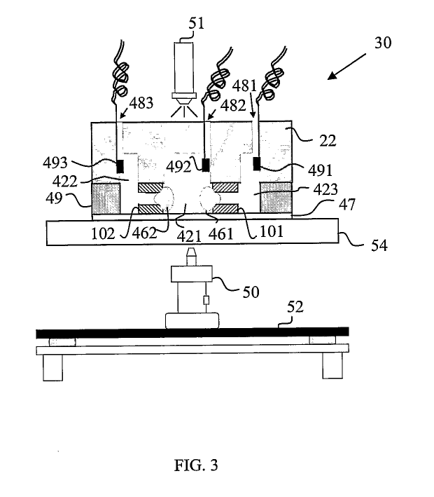

FIG. 3 shows a simplified diagram of a setup using a microfluidic device 30 according to an embodiment of the invention for viewing under a microscope. The device 30 comprises a central first fluid chamber 421 that is separated from second fluid chambers 422, 423 by partitioning elements 101, 102, respectively. A first cell 461 is immobilised over the inlet of partitioning element 101, while a second cell 462 is immobilised over the inlet of partitioning element 102. The glass cap 22 is provided with ports 481, 482, 483 through which electrodes 491, 492, 493 connected to an external measurement device are inserted and accesses fluid chambers 421, 422, 423 in the device 30 for making electrical measurements of such as ion currents through or voltage potential across the cell. The entire device 30 rests on a transparent platform 54, with etch stop layer 47 facing the transparent platform. Observation is carried out using an inverted microscope 50 with an illumination source 51 arranged above the device 30. The microscope 50 is arranged on a vibration isolation table 52.

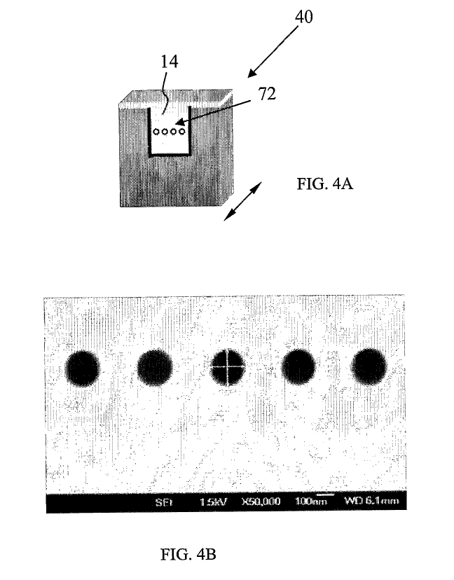

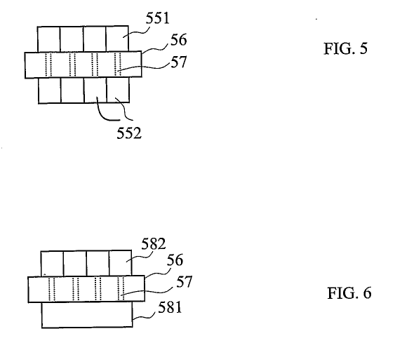

FIG. 4A and FIG. 4B shows an embodiment of the invention in which partitioning element 40 comprises a plurality of channels 72 formed in the filler member 14. The plurality of channels 72 enables more than 1 biological entity to be immobilised on a single partitioning element, if desired. As can be seen in FIG. 4B, each channel is formed in a respective recess. In a further embodiment, this partitioning element is connected to an array of first fluid chambers 551 (see FIG. 5) and a respective array of second fluid chambers 552 via channels 57 arranged within partitioning elements 56. In this configuration, a large quantity of drugs, for example, can be screened for efficacy simultaneously. Alternatively, a single (common) first fluid chamber 581 may be present in the device for receiving a sample (see FIG. 6). The first fluid chamber 581 is fluidly connected to an array of second fluid chambers 582 via channels 57 arranged within partitioning elements 56. In this configuration, there only one common ground electrode needs to be located in the first fluid chamber, and as many independent sensing electrodes as the number of the second fluid chambers are disposed in each isolated second fluid chambers.

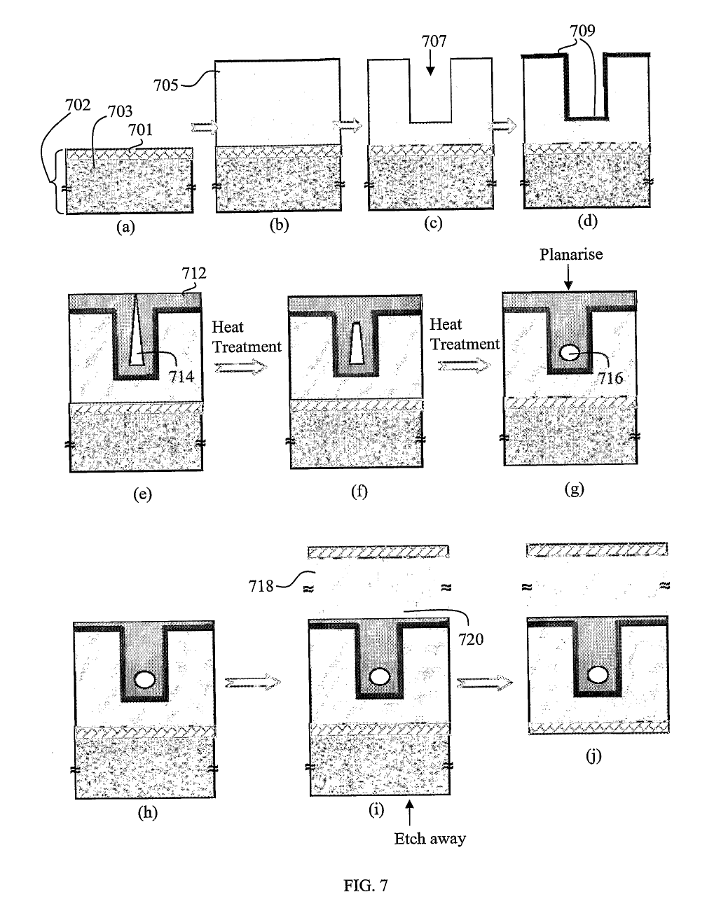

The general process for fabricating a channel element as shown in FIG. 7 starts with a handling substrate 702, such as a conventional silicon wafer comprising a silicon layer 703 having arranged thereon an silicon-etch stop layer 701 (FIG. 7a). Typically, the etch stop layer comprises thermal oxide of about 100 nm thickness with 150 nm silicon nitride. Thick, optically transparent layer of about 4 μm of silicon oxide 705 (FIG. 7b) is deposited on the etch stop layer 701. Then, trenches 707 are etched in the silicon oxide (FIG. 7c), followed by deposition of a separation layer 709 (for example, 100 nm poly-silicon or silicon nitride, FIG. 7d). Trenches are then partially filled (FIG. 7e) with a doped silicon oxide layer 712 (such as PSG) in such a manner that a void 714 is laterally formed in the doped silicon oxide layer 712. After heat treatment, the void squeezes (FIG. 7f) and finally is re-shaped into a circular channel 716 (FIG. 7g). The doped silicon oxide layer is then planarised by grinding or etching (FIG. 7g and FIG. 7h). Fluid chambers are subsequently defined and etched in the silicon oxide layer, with the channel element forming a fluid connection between chambers. A capping substrate, such as a transparent substrate in the form of a glass cover, is arranged over the planarised doped silicon oxide layer to cover the opening of each etched fluid chamber. In this example, a glass cover 718 is bonded to the planarised surface of the doped silicon oxide layer. This can be accomplished by anodic, fusion or adhesive bonding, to name a few examples. The glass cover 718 may further include a depression 720 that is shaped according to the opening to the fluid chamber that has been etched in the silicon oxide (FIG. 7i). Mechanical and chemical processes can be used to etch away the opaque silicon layer beneath the etch stop layer in order to obtain a fully transparent device (FIG. 7j).

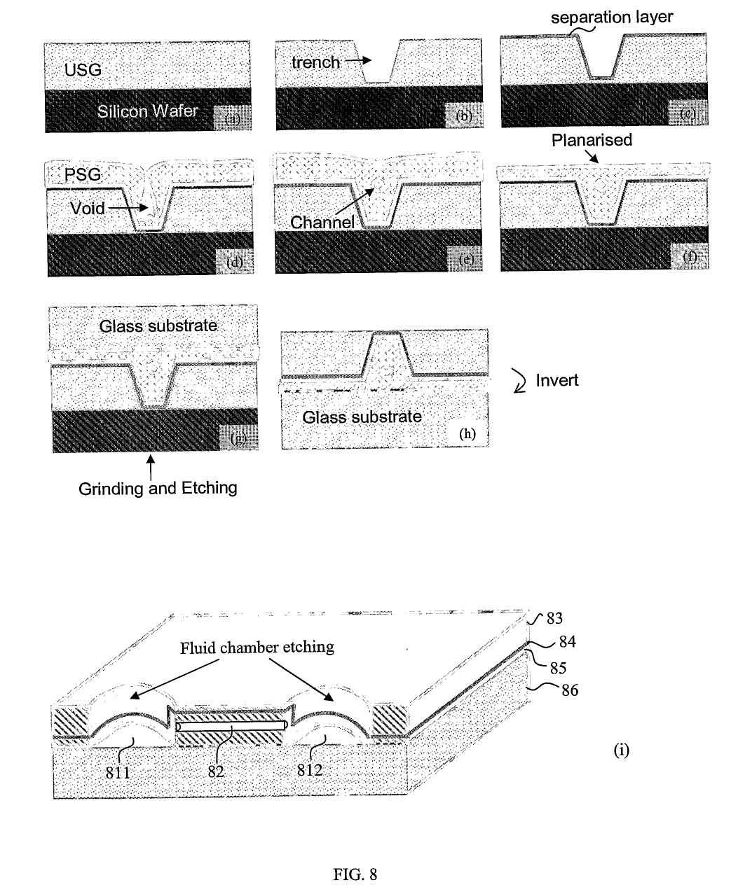

FIG. 8A to FIG. 8H depict a particular fabrication procedure which was carried out according to the general process described above for FIG. 7. Firstly, a 4 μm to 10 μm thick silicon oxide (SiO₂) or undoped silica glass (USG) was deposited onto a handling substrate comprising a silicon wafer through plasma-enhanced chemical vapour deposition (PECVD). A trench was created in the deposited SiO₂ or USG during reactive ion etching (RIE) process after patterning with standard lithography steps. Subsequently, a thin transparent silicon nitride dielectric layer was deposited into the trench. Phosphorus silica glass (PSG) was used as filling material and was deposited by PECVD onto the trenches, giving rise to non-conformal deposition and forming (triangular) voids within the trenches. The structure was subjected to thermal annealing at the reflow temperature of the filling material; circular micro/nano channels are formed within the trenches. The uneven surface topography was subsequently planarized using chemical mechanical polishing tool (CMP). Cleaning on the planarised surface was done with Piranha solution. A capping substrate comprising a glass substrate was bonded to the planarised surface (e.g. by anodic, fusion or adhesive bonding). Removal of silicon wafer substrate by a combination of mechanical backside grinding and selective TMAH (tetramethyl ammonium hydroxide) or KOH wet etching on the silicon oxide layer was carried out. The Si etch stops at the silicon dioxide-silicon wafer interface due to the presence of the etch stop layer.

FIG. 8I shows a cross section of the structure formed from the fabrication. Two separate fluid chambers 811 and 812 are etched into the structure from the undoped silica glass (USG) surface, both fluid chambers being arranged to be fluidly connected by the small microfluidic channel 82. The USG layer 83 is separated from the filler member 85 by a silicon nitride layer (separation layer) 84. The glass platform 86 acts as a base for the fluid chambers 811, 812. It is to be noted that open fluid chambers can be fabricated after the step depicted in FIG. 8F prior to the bonding process.

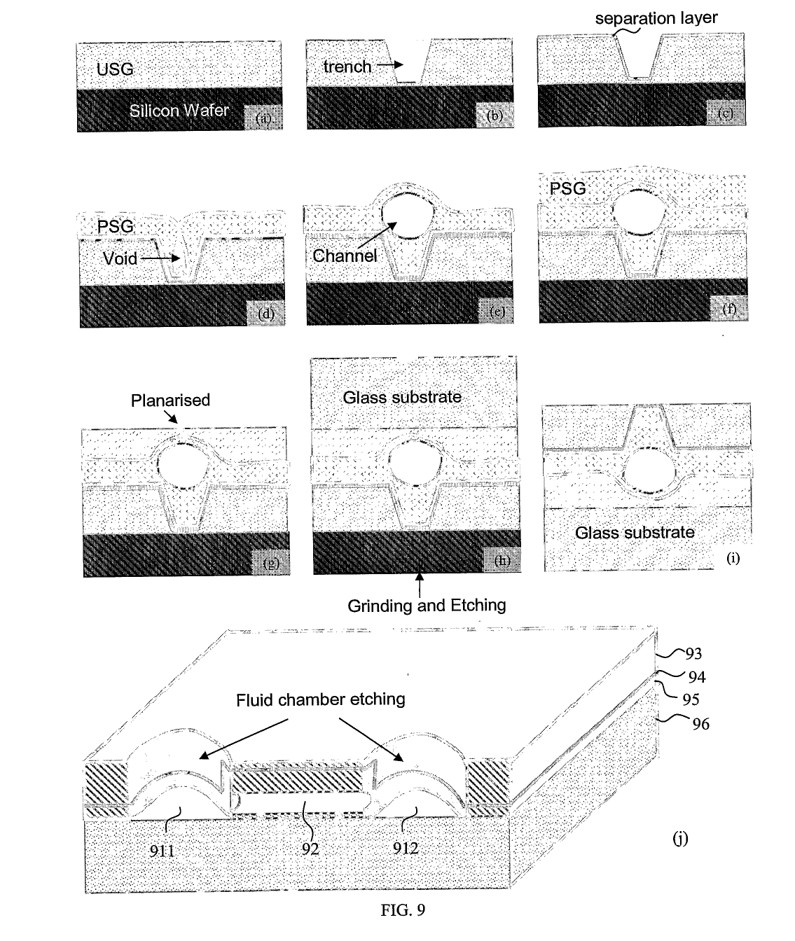

FIG. 9A to FIG. 91 depicts another procedure which was carried out according to the general process described in FIG. 7. This procedure resulted in channels with diameters larger than the width of the recess, so the channels were located partially outside of the recess. Firstly, a 4 μm to 10 μm thick silicon oxide (SiO₂) or undoped silica glass (USG) was deposited onto a silicon wafer through PECVD. A trench was created during RIE etching process after patterning with standard lithography steps. A thin transparent, highly stressed film, such as silicon nitride or standard poly-silicon, was deposited into the trench. A Phosphorus Silica Glass (PSG) filling material was deposited by PECVD into the trench, giving rise to non-conformal surface topography in which elongated voids, which could be elliptical or triangular in cross-section, were formed within the trenches. The base substrate is subjected to thermal annealing at the reflow temperature of the filling material; circular microchannels bloated above the surface of the filling material. PECVD or HDP (High Density Plasma) deposition systems can be used to deposit another layer of PSG/USG (un-doped silicate glass) on the uneven topography to even up the protruding portion and to strengthen the side walls of the microchannels. The uneven surface topography is planarized using chemical mechanical planarization tool (CMP). Device wafer is then cleaned in Piranha solution before being anodically bonded to a glass substrate. Removal of silicon substrate is carried out by backside grinding and selective TMAH (tetramethyl ammonium hydroxide) etch towards the silicon oxide layer.

FIG. 9J shows a cross section of the structure formed from the fabrication. Two separate fluid chambers 911 and 912 are etched into the structure from the undoped silica glass (USG) surface, both fluid chambers being arranged to be fluidly connected by the large microfluidic channel 92. The USG layer 93 is separated from the filler member 95 by a silicon nitride layer (separation layer) 94. The glass platform 96 acts as a base for the fluid chambers 911, 912. It is to be noted that open fluid chambers can be fabricated after the step depicted in FIG. 9G prior to the bonding process.

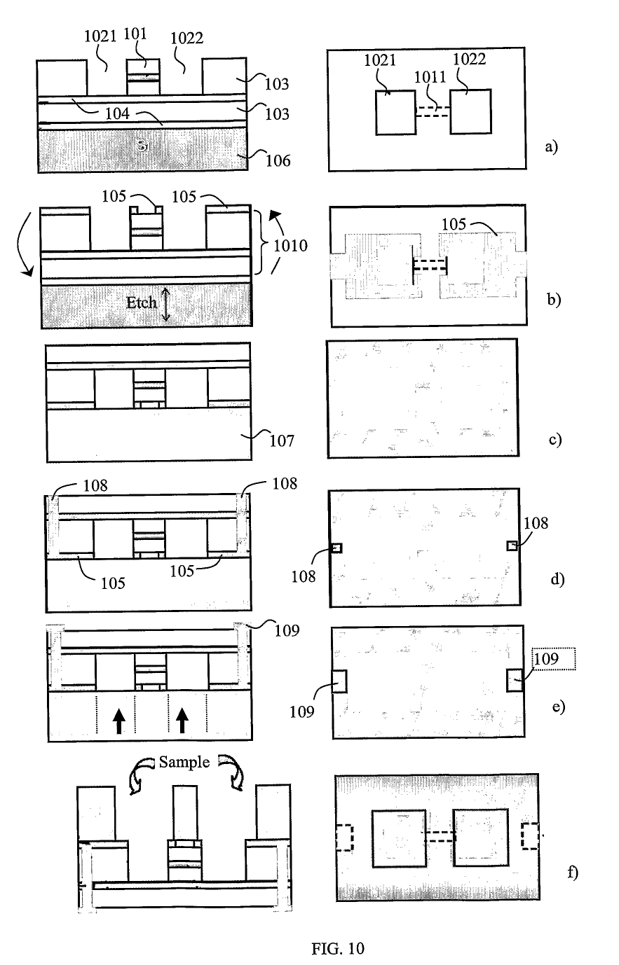

FIG. 10 depicts from a cross-sectional view the steps required for forming a device shown in FIG. 2. After the formation of a channel element 101, fluid chambers 1021 and 1022 are etched into the base substrate 103 in a manner such that each end of the channel 1011 in the channel element opens up into a fluid chamber 1021 or 1022. A silicon nitride separation layer 104 is depicted as being disposed between the channel element 101 and the base substrate 103. Electrical contacts 105 are formed on the surface of the base substrate, surrounding the opening of each fluid chamber 1021, 1022 (FIG. 10b). After a transparent platform 107 (such as a glass cover) is bonded on the other side of the structure, the silicon layer 106 beneath the base substrate 103 is etched away. The transparent platform is bonded to the base substrate via anodic bonding to seal the fluid chambers (FIG. 10c). Vias 108 were etched into the transparent platform to expose a portion of the covered electrical contacts 105, so that sensing electrodes can be inserted therein to contact the electrical contacts 105 for making measurements (FIG. 10d). Subsequently, pads 109 were formed over the vias 108. Entrances into the fluid chambers 1021, 1022 were fabricated by etching away the portion of the glass platform over the fluid chamber 1021, 1022 to form a finished product.

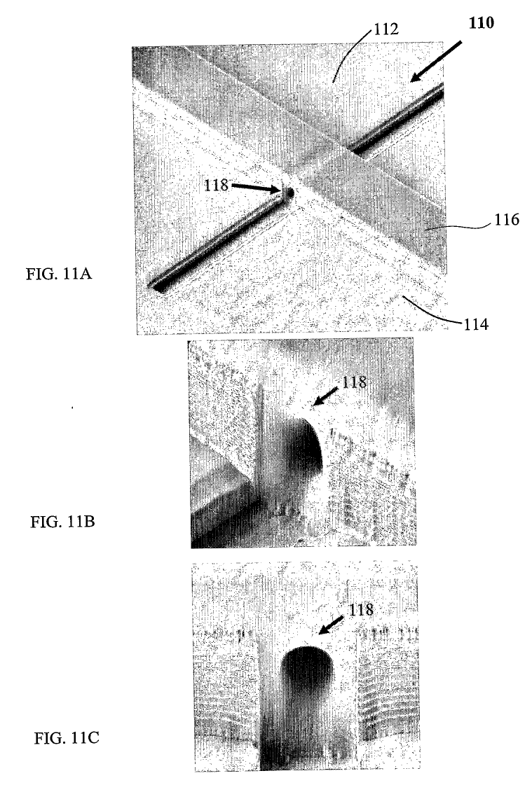

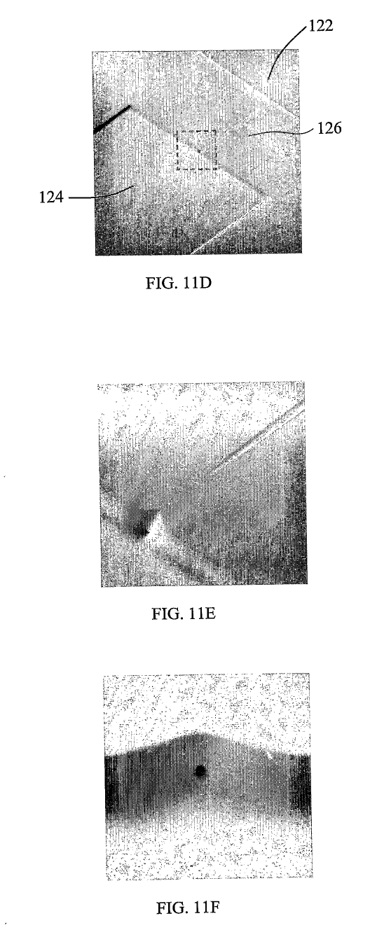

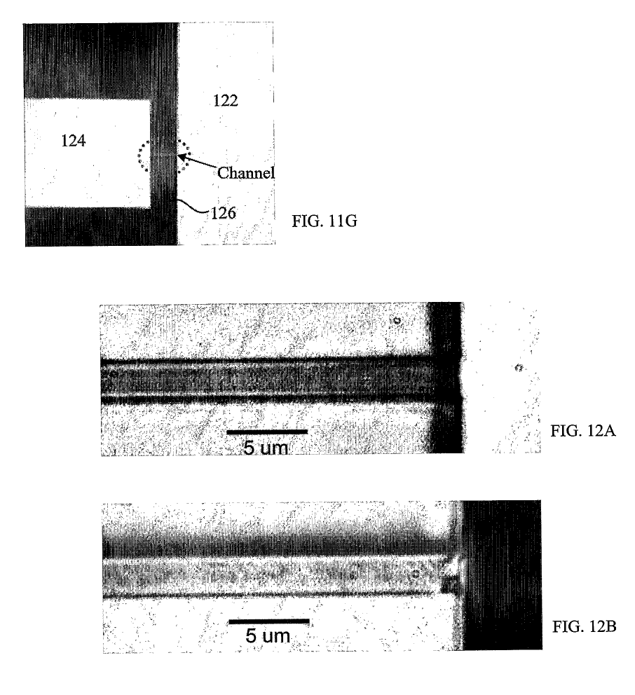

FIG. 11A shows a perspective view of an actual completed device 110 having fluid chambers 112, 114 and partitioning element 116 with a channel 118 buried therein. FIG. 11B and FIG. 11C show close up views of the opening of the channel, which is seen to be substantially circular. FIG. 11D shows a perspective view of another structure with channel diameter of about 300 nm prior to bonding of a glass cover from a magnified SEM image. Reservoir inlet 122 and reservoir outlet 124 are separated by a channel element 126. FIG. 11E shows a close up view of the portion identified by a dotted lines in FIG. 11D. FIG. 11F shows a highly magnified image of the entrance of the channel in the partitioning element. As can be seen from the figure, channels with substantially circular inlet/outlets can be fabricated even at very small dimensions. FIG. 11G shows the top view of the embodiment depicted in FIG. 11D. Capillary action leading to the movement of fluorescent dye from the reservoir inlet 124 to the reservoir outlet 122 was observed. The absence of fluorescent dye under the channel element showed that anodic bonding was sufficient to prevent fluid leakage within the device.

FIG. 12A is a magnified optical image of a portion of a 1.5 micrometer wide channel in a channel element under Transmission Mode of 100×. Under this mode, the reservoir outlet containing fluid is seen to be transparent in the image, while the channel appears dark and shaded. In the actual colour image, the entire structure appears light orange, with the microchannel appearing darker than the other structures. FIG. 12B is a magnified optical image of a portion of the channel in a channel element under Reflection Mode of 100×. Under this mode, the reservoir containing fluid appears black in the image, while the channel appears lightly shaded. In the actual colour image, the entire structure appears light green, with the microchannel appearing darker than the other structures. It is suggested that the translucence of the device as seen in the images depend on the thickness of the separation layer.

In a fabrication example to demonstrate the formation of a circular channel in transparent materials, a variety of process parameters of temperature and pressure were chosen. Experiments were carried out at different process conditions, 6 typical conditions are shown in the Table 1 using BPSG and PSG as the filler member:

TABLE 1

| | Channel material & | Reflow temperature | |
|---|---|---|---|
| | Deposition Pressure | (° C.) | Time (min.) |
| 1. | BPSG at 50 Torr | 900 | 240 |
| 2. | BPSG at 50 Torr | 950 | 120 |
| 3. | BPSG at 50 Torr | 1000 | 40 |
| 4. | PSG at 3 Torr | 1050 | 120 |
| 5. | PSG at 3 Torr | 1100 | 45 |
| 6. | PSG at 3 Torr | 1150 | 30 |

Base members were etched in accordance with known micromachining techniques to form a recess (see for example J. Microelectromech. Sys. Vol. 5, No. 4, December 1996). The straight walls and high aspect ratio trenches are achieved through reactive ion etching (RIE) techniques.

Trench sizes of less than 0.2 μm to 3 μm wide and less then 0.5 μm to 7 μm deep were fabricated according to the above protocol. It is to be pointed out that trenches with smaller or larger dimensions may be required for different target dimensions of the channels. PECVD was used to fill doped silicon dioxide (PSG) into the trenches at low pressure (2.5 T). A 2.27 μm high and 0.99 μm wide channel in silicon dioxide with silicon nitride (Si₃N₄) as the separation layer was obtained. A 4 μm thick extraneous PSG layer, which is part of the filler member, is present over the top surface of the base substrate. Modelling of micro/nano-channel cross section dimension is carried out as follows. Let the non-conformal silicon oxide be filled in the trenches at temperature Ti and pressure Pi. The void in the trench has a cross sectional area Ai. Since the void created in the trench is at sub-atmospheric pressure, the void has tendency to reduce if the silicon oxide is softened. Depending on the softening conditions, the final dimension (Af) of the void can be predicted. If the softening is done at temperature Tf and pressure Pf, then from ideal gas law the following equation applies:

(Pi·Vi)/Ti=(Pf·Vf)/Tf (1)

where, Vi and Vf are the initial and final volume of the void.

But since the length of the void (and trench) remains unchanged, Vi and Vf can be replaced by Ai and Af respectively in (1) to arrive at

(Pi·Ai)/Ti=(Pf·Af)/Tf (2)

or,

Af=(Pi/Pf)·(Tf/Ti)·Ai (3)

In a typical case, BPSG may be deposited at 400° C. and 50 Torr pressure. Under such conditions, it is observed that a void of about 6 μm² (6.0 μm×1.0 μm) cross-sectional area is created in the 2 μm wide and about 7.7 μm deep trench. This void can be deformed to circular cross sections after exposure to heating under pressure. Various examples of the channels obtained through this method is summarised in Table 2.

TABLE 2

| | Initial cross- | | Final cross- | | Actual |
|---|---|---|---|---|---|
| | sectional | Reflow | sectional | Radius of | radius of |
| S. | area | temperature | area (A_{f}), in | channel, | channel, |
| No. | (Aᵢ), in μm² | (° C.) | μm² | (in μm) | (in μm) |
| 1 | 6.0 | 900 | 0.688 | 0.467 | 0.406 |
| 2 | 6.0 | 950 | 0.717 | 0.477 | 0.433 |
| 3 | 6.0 | 1000 | 0.746 | 0.487 | 0.505 |

In summary, the present invention is capable of producing lateral channels with circular cross-section in transparent materials, providing the minimum surface/frictional resistance and better electrical sealing for optical applications. These channels have cross-sectional diameter in the range of several microns to tens of nanometres. The channel cross section dimensions can be predicted and controlled precisely by varying fabrication conditions. The fabrication processes are fully CMOS compatible and can therefore be implemented at existing silicon foundries. Channel fabrication cost is low as no specialized tools/processes such as electron beam lithography, laser source, polymers, etc. are used. The invention can also be used to fabricate multiple, self-aligned channels, both laterally and vertically.

Although this invention has been described in terms of preferred embodiments, it has to be understood that numerous variations and modifications may be made, without departing from the spirit and scope of this invention as set out in the following claims.

1. A microfluidic device comprising:
a substantially transparent base substrate having a recess defined therein by at least two opposing lateral walls and a base wall,
a substantially transparent filler member having at least a portion thereof occupying the recess,
a substantially transparent separation layer disposed between the base substrate and the filler member such that the entire filler member is separated from the base substrate, and a channel defined within the filler member,
wherein
the channel comprises an inlet and an outlet, said inlet being arranged on a first lateral wall of the filler member, and said outlet being arranged on a second lateral wall of the filler member,
the first lateral wall of the filler member being arranged in opposing relationship with the second lateral wall of the filler member, and
at least a portion of the first and the second lateral walls of the filler member being at least substantially perpendicular to the opposing lateral walls defining the recess.

2.-4. (canceled)

5. The microfluidic device of claim 1, wherein the inlet of the channel is at least substantially circular in shape.

6.-8. (canceled)

9. The microfluidic device of claim 1, wherein the channel has a length of between about 1 micrometers to about 100 micrometers.

10. (canceled)

11. The microfluidic device of claim 1, wherein the base substrate is selected from the group consisting of silicon dioxide and transparent alumina.

12.-16. (canceled)

17. The microfluidic device of claim 1, wherein the separation layer is selected from a silicon-based ceramic material, polysilicon and amorphous silicon.

18. (canceled)

19. The microfluidic device of claim 1, wherein the filler member comprises doped silicon oxide.

20. (canceled)

21. The microfluidic device of claim 1, further comprising
at least a first fluid chamber and a second fluid chamber, the first fluid chamber being fluidly connected to the second fluid chamber via the channel that is defined in the portion of the filler member occupying the recess.

22. The microfluidic device of claim 21, wherein the first fluid chamber and the second fluid chamber are monolithically defined in the base substrate.

23. (canceled)

24. The microfluidic device of claim 1, further comprising
a plurality of recesses defined in the substrate,
the filler member having corresponding portions thereof arranged in each recess, and
a channel arranged along each recess.

25. A microfluidic device of claim 1, further comprising:
a plurality of first fluid chambers and a plurality of second fluid chambers, each first fluid chamber being fluidly connected with a corresponding second fluid chamber via at least one of said plurality of channels.

26. The microfluidic device of claim 21, wherein at least one of said first and second fluid chambers operably connected to at least one microfluidic unit operation module.

27. (canceled)

28. The microfluidic device of claim 21, further comprising a sensing electrode disposed in the first fluid chamber and a reference electrode disposed in the second fluid chamber.

29. (canceled)

30. The microfluidic device of claim 21, further comprising a capping substrate for covering said at least first fluid chamber and second fluid chamber.

31. (canceled)

32. A microfluidic device for analysing a particle comprising:
at least a first and a second fluid chamber defined in a substantially transparent base substrate, the second fluid chamber being fluidly connected to the first fluid chamber by a channel element defined in the base substrate, said channel element comprising:
a recess defined in the base substrate by at least two opposing lateral walls and a base wall, said recess extending between the first fluid chamber and the second fluid chamber,
a substantially transparent filler member having at least a portion thereof occupying the recess,
a substantially transparent separation layer arranged between the base substrate and the filler member such that the entire filler member is separated from the base substrate, and
a channel defined in the filler member,
wherein the channel comprises an inlet arranged on a first lateral wall of the filler member, and an outlet arranged on a second lateral wall of the filler member, said first lateral wall of the filler member being arranged in opposing relationship with the second lateral wall of the filler member, and
at least a portion of said first lateral wall and said second lateral wall of the filler member being at least substantially perpendicular to the opposing lateral walls defining the recess.

33. The microfluidic device of claim 32, wherein the channel is arranged along the recess.

34. The microfluidic device of claim 32, wherein the channel is arranged within the recess.

35. The microfluidic device of claim 32, wherein a portion of the channel is arranged outside the recess.

36. The microfluidic device of claim 32, wherein both the inlet and the outlet of the channel are is at least substantially circular in shape.

37. The microfluidic device of claim 36, wherein the diameter of the inlet and the outlet is between about 0.1 micron to about 20 micron.

38. The microfluidic device of claim 32, wherein the channel is at least substantially cylindrical in shape.

39. (canceled)

40. The microfluidic device of claim 32, further comprising a plurality of channels arranged in the filler member.

41. The microfluidic device of claim 32, further comprising a further fluid chamber that is fluidly connected to at least one of said first and said second fluid chambers via a further channel element

42. (canceled)

43. The microfluidic device of claim 32, wherein an electrical measurement device is operably connected to the first fluid chamber and the second fluid chamber for determining an electrical characteristic of a particle that is placed in either fluid chamber.

44. The microfluidic device of claim 32, further comprising a capping substrate for covering said at least first and second fluid chambers.

45. (canceled)

46. A method of forming a microfluidic device, comprising:
providing a substantially transparent base substrate,
forming a recess in a surface of the base substrate,
forming a substantially transparent separation layer on said surface of the base substrate,
filling said recess with a substantially transparent filling material, and
subjecting the filling material to a condition that causes it to deform such that a channel is formed in the filling material,
wherein the separation layer is disposed between the base substrate and the filler member such that the entire filler member is separated from the base substrate.

47.-58. (canceled)

59. A method of analyzing the status of a biological entity, comprising:
introducing the biological entity into the first fluid chamber of a microfluidic device as defined in claim 32,
measuring a first (reference) electrical signal that is associated with a first status of the biological entity,
exposing the biological entity to a condition that is suspected to be capable of changing the state of the biological entity, and
measuring a second electrical signal that is associated with the status of the biological entity after exposure to said condition.

60.-74. (canceled)
